(19) United States
(12) Patent Application Publication
(10) Pub. No.: US 2006/0063718 A1
(43) Pub. Date: Mar. 23, 2006

(54) Topical glutathione compositions

(76) Inventor: Nicholas V. Perricone, Meriden, CT (US)

Correspondence Address: ST. ONGE STEWARD JOHNSTON & REENS, LLC, 986 BEDFORD STREET, STAMFORD, CT, 06905-5619, US

(21) Appl. No.: 11/272,842

(22) Filed: Nov. 14, 2005

Related U.S. Application Data
(62) Division of application No. 10/789,233, filed on Feb. 27, 2004.

(51) Int. Cl. A61K 38/05; A61K 31/385

(52) U.S. Cl. 514/18; 514/440

(57) ABSTRACT

A composition for treatment of inflammatory skin conditions is provided having a carrier; reduced glutathione; and alpha lipoic acid. The composition preferably contains 25% to 60% by weight glutathione; and 0.5% to 5% by weight alpha lipoic acid. The composition is useful for topical treatment of psoriasis and other inflammatory skin diseases.

### PRIOR APPLICATIONS

The present application is a divisional application of a co-pending U.S. patent application Ser. No. 10/789,233, filed Feb. 27, 2004, entitled “Topical Glutathione Treatments.”

### FIELD OF THE INVENTION

The present invention methods for the treatment of inflammatory skin conditions.

### BACKGROUND OF THE INVENTION

Psoriasis is a lifelong skin disease that occurs when faulty signals in the immune system cause keratinocyte skin cells to regenerate too quickly, on the order of every three to four days instead of the usual 30-day cycle. Extra skin cells build up on the skin's surface, forming red, flaky, scaly lesions that can itch, crack, bleed and be extremely painful. Psoriasis generally involves the joints, limbs and scalp but it can appear anywhere on the body, covering some people from head to toe. More than 5 million Americans have been diagnosed with psoriasis and/or psoriatic arthritis, a degenerative disease of the joints and connective tissues associated with psoriasis. Psoriasis typically first strikes people between the ages of 15 and 35, but can affect anyone at any age, including children.

Psoriasis is characterized by erythematous eruptions, often in papules or plaques, and usually having a white, silvery scale. Psoriasis is generally considered an inflammatory skin condition. Other inflammatory skin conditions include atopic dermatitis (eczema), seborrhoeic dermatitis, rosacea, acne, as well as contact dermatitis (typically arising from allegic reaction to poison ivy and other allegens).

Conventional therapeutic regimens for psoriasis include topical or intralesional application of corticosteroids, anthralin, tazarotene (a retinoid), calcipotriene (vitamin D3) and/or zinc compounds, and/or selenium compounds, and/or coal tar compounds; or various light therapies; or an oral or injected systemic agent. No single therapy is ideal, and it is rare for a patient not to be treated with several alternatives during the relapsing and remitting course of the disease. Other inflammatory skin conditions are typically treated with the same types of therapies.

As set forth in more detail hereafter, the present invention is based on the topical use of glutathione as a treatment for psoriasis and other inflammatory skin conditions. Reduced glutathione, most commonly called glutathione or GSH, is a relatively small molecule found in animals and plants. GSH is a water-phase orthomolecule. GSH is the smallest intracellular thiol molecule. Its high electron-donating capacity combined with high intracellular concentration generate great reducing power. Glutathione is thus recognized as a potent antioxidant and enzyme cofactor and for a critical role in regulating cell activity.

Reduced glutathione (GSH) is a linear tripeptide of L-glutamine, L-cysteine, and glycine. Technically, N-L-gamma-glutamyl-cysteinyl glycine or L-glutathione, the molecule has a sulfhydryl (SH) group on the cysteinyl portion, which accounts for its strong electron-donating character. As electrons are lost the molecule becomes oxidized, and two such molecules become linked (dimerized) by a disulfide bridge to form glutathione disulfide or oxidized glutathione (GSSG). This linkage is reversible upon re-reduction. GSH is under tight homeostatic control both intracellularly and extracellularly. A dynamic balance is maintained between GSH synthesis, its recycling from GSSG/oxidized glutathione, and its utilization.

GSH synthesis involves two closely linked, enzymatically controlled reactions that utilize ATP. First cysteine and glutamate are combined, by gamma-glutamyl cysteinyl synthetase. Second, GSH synthetase combines gamma-glutamylcysteine with glycine to generate GSH. As GSH levels rise, they self-limit further GSH synthesis; otherwise, cysteine availability is usually rate-limiting. Fasting, protein-energy malnutrition, or other dietary amino acid deficiencies limit GSH synthesis.

GSH recycling is catalyzed by glutathione disulfide reductase, which uses reducing equivalents from NADPH to reconvert GSSG to 2GSH. The reducing power of ascorbate helps conserve systemic GSH. GSH is used as a cofactor by (1) multiple peroxidase enzymes, to detoxify peroxides generated from oxygen radical attack on biological molecules; (2) transhydrogenases, to reduce oxidized centers on DNA, proteins, and other biomolecules; and (3) glutathione S-transferases (GST) to conjugate GSH with endogenous substances (e.g., estrogens) and to exogenous electrophiles (e.g., arene oxides, unsaturated carbonyls, organic halides), and diverse xenobiotics.

Free radical and other oxidative agents can deplete GSH. The homeostatic glutathione redox cycle attempts to maintain GSH levels as it is being consumed. Amounts available from foods are limited (less than 150 mg/day), and oxidative depletion can outpace synthesis.

The liver is the largest GSH reservoir. The parenchymal cells synthesize GSH for P450 conjugation and numerous other metabolic requirements, then export GSH as a systemic source of SH/reducing power. GSH is carried in the bile to the intestinal luminal compartment. Epithelial tissues of the kidney tubules, intestinal lining, and lung, have substantial P450 activity and modest capacity to export GSH.

GSH equivalents circulate in the blood predominantly as cystine, the oxidized and more stable form of cysteine. Cells import cystine from the blood, reconvert it to cysteine (likely using ascorbate as cofactor), and from it synthesize GSH. Conversely, inside the cell GSH helps re-reduce oxidized forms of other antioxidants such as ascorbate and alpha-tocopherol.

GSH is an extremely important cell protectant. It directly quenches reactive hydroxyl free radicals, other oxygen-centered free radicals, and radical centers on DNA and other biomolecules. GSH protects skin, lens, cornea, and retina against radiation damage, and the biochemical foundation of P450 detoxication in the liver, kidneys, lungs, intestinal epithelia, and other organs.

GSH is the essential cofactor for many enzymes which require thiol-reducing equivalents, and helps keep redox-sensitive active sites on enzymes in the necessary reduced state. Higher-order thiol cell systems the metallothioneins, thioredoxins, and other redox regulator proteins are ultimately regulated by GSH levels and the GSH/GSSG redox ratio.

GSH and its metabolites also interface with energetics and neurotransmitter syntheses, through several prominent metabolic pathways. GSH availability down-regulates the pro-inflammatory potential of leukotrienes and other eicosanoids.

GSH levels in human tissues normally range from 0.1 to 10 millimolar (mM), most concentrated in the liver (up to 10 mM) and in the spleen, kidney, lens, erythrocytes, and leukocytes. Plasma concentration is in the micromolar range (approx. 4.5 μM). Oxidative stressors that can deplete GSH include ultraviolet and other radiation; viral infections; environmental toxins, household chemicals, and heavy metals; surgery, inflammation, burns, septic shock; and dietary deficiencies of GSH precursors and enzyme cofactors.

### SUMMARY OF THE INVENTION

The primary object of this invention is to provide a treatment for psoriasis and other inflammatory conditions of the skin, and more particularly, to provide a therapy based upon topical application to affected skin areas of an active form of glutathione, or precursors thereof, preferably in association with a dermatologically acceptable carrier or vehicle. In the preferred embodiments of the invention, the glutathione is provided in a carrier at very high concentration levels, in the range of 16-70 percent by weight, more preferably 35-60 percent by weight.

### DETAILED DESCRIPTION OF THE INVENTION

Topical compositions containing active forms of glutathione according to the present invention are topically applied to and absorbed by the skin tissue. Generally, topical application to skin tissue is accomplished in association with a carrier, and particularly one in which the glutathione is soluble per se or is effectively solubilized (e.g., as an emulsion or microemulsion). Where employed, the carrier is inert in the sense of not bringing about a deactivation or oxidation of the glutathione active ingredient(s), and in the sense of not bringing about any adverse effect on the skin areas to which it is applied.

It is expected that in all cases, glutathione will be preferably applied in its reduced form GSH as this is expected to be the most active form of glutathione. However, other forms of glutathione having the requisite activity can also be used.

Topical administration of glutathione avoids the problem of the breakdown into constituent components that occurs in oral administration of glutathione. Thus is should not be necessary to use glutathione precursors to obtain the desired beneficial effect of the gultathione. Nevertheless, there is some possibility that glutathione precursors would also be effective for use in the invention, and thus the invention herein shall also include use of glutathione precursors such as cystine or cysteine, with or without the additional compounds glutamine/glutamate and/or glycine.

In one preferred practice of the invention, glutathione will be applied in admixture with the dermatologically acceptable carrier or vehicle (e.g., as a lotion, cream, ointment, soap, stick, or the like) so as to facilitate topical application and, in some cases, provide additional therapeutic effects as might be brought about, e.g., by moisturizing of the affected skin areas. While the carrier for the topical composition can consist of a relatively simple solvent or dispersant such as water, it is generally preferred that the carrier comprise a composition more conducive to topical application, and particularly one which will form a film or layer on the skin to which it is applied so as to localize the application and provide some resistance to washing off by immersion in water or by perspiration and/or aid in the percutaneous delivery of the active agent(s). Many preparations are known in the art, and include lotions containing oils and/or alcohols and emollients vegetable oils, hydrocarbon oils and waxes, silicone oils, animal or marine fats or oils, glyceride derivatives, fatty acids or fatty acid esters, or alcohols or alcohol ethers, lecithin, lanolin and derivatives, polyhydric alcohols or esters, wax esters, sterols, phospholipids and the like, and generally also emulsifiers (nonionic, cationic or anionic), although some of the emollients inherently possess emulsifying properties. In the preferred embodiment, the carrier is a phospholipid, most preferably, lecithin.

As noted, these ingredients can be formulated into a cream, lotion, or gel, or a solid stick, by utilization of different proportions of the ingredients and/or by inclusion of thickening agents such as gums or other forms of hydrophilic colloids. One possible embodiment is a solution used to saturate a pad used to wipe affected areas; another is a cleanser; and others are lotions, creams, and gels, which are referred to herein as dermally or dermatologically acceptable carriers, and are formulated using conventional techniques known to those of ordinary skill in the art. The term “topical composition” as used herein shall mean the complete product including the glutathione active ingredient, the carrier, and any adjuvants, thickeners, excipients, etc. as described herein which is applied to a person's skin.

The quantity of the glutathione active ingredient in the carrier may be varied or adjusted widely depending upon the particular application, the potency of the particular compound, the desired concentration. Generally, it is contemplated that the present invention will deliver glutathioine to the skin at very high concentrations. The quantity of the glutathione active ingredient will range between 16% to 70% by weight of the topical composition. In more potent embodiments, the quantity of glutathione active ingredient will range between 35% to 70% by weight of the topical composition. In a lower potency embodiment, the quantity of glutathione active ingredient will range between 16% to 35% by weight of the topical composition. In another embodiment, the quantity of glutathione active ingredient will range between 20% to 35% by weight of the topical composition. In another embodiment, the quantity of glutathione active ingredient will range between 35% to 60% by weight of the topical composition.

Generally in the practice of methods of the invention, the topical composition is topically applied to the skin areas, such as that of the face, at predetermined intervals often as a moisturizer, tinted foundation, cleanser, toner, lotion, cream, or gel, it generally being the case that gradual improvement is noted with each successive application.

The topical composition of the invention can contain additional ingredients commonly found in skin care compositions and cosmetics, such as, for example, tinting agents, emollients, skin conditioning agents, emulsifying agents, humectants, preservatives, antioxidants, perfumes, chelating agents, etc., provided that they are physically and chemically compatible with other components of the composition. Preservatives include, but are not limited to, C₁-C₃ alkyl parabens and phenoxyenthanol, typically present in an amount ranging from about 0.5% to about 2.0% by weight percent, based on the total composition. Emollients, typically present in amounts ranging from about 0.01% to 5% of the total composition can include, but are not limited to, fatty esters, fatty alcohols, mineral oils, polyether siloxane copolymers, and mixtures thereof. Humectants, typically present in amounts ranging from about 0.1% to about 5% by weight of the total composition include, but are not limited to, polyhydric alcohols such as glycerol, polyalkylene glycols (e.g., butylene glycol, propylene glycol, dipropylene glycol, polypropylene glycol, and polyethylene glycol) and derivatives thereof, alkylene polyols and their derivatives, sorbitol, hydroxy sorbitol, hexylene glycol, 1,3-dibutylene glycol, 1,2,6-hexanetriol, ethoxylated glycerol, propoxylated glycerol, and mixtures thereof. Emulsifiers can be typically present in amounts from about 1% to about 10% by weight of the composition, and include, but are not limited to, stearic acid, cetyl alcohol, stearyl alcohol, steareth 2, steareth 20, acrylates/C10-30 alkyl acrylate cross-polymers, and mixtures thereof. Chelating agents, typically present in amounts ranging from about 0.01% to about 2% by weight, include, but are not limited to, ethylenediamine tetraacetic acid (EDTA) and derivatives and salts thereof, dihydroxyethyl glycine, tartaric acid, and mixtures thereof.

Antioxidants for the composition can be present in an amount ranging from about 0.02% to about 0.5% by weight of the composition, include, but are not limited to, butylated hydroxy toluene (BHT); vitamin C and/or vitamin C derivatives, such as fatty acid esters of ascorbic acid, particularly asocorbyl palmitate; butylated hydroanisole (BHA); phenyl-α-naphthylamine; hydroquinone; propyl gallate; nordihydroquiaretic acid; vitamin E and/or derivatives of vitamin E, including tocotrienol and/or tocotrienol derivatives; calcium pantothenates; green tea extracts; mixed polyphenols; and mixtures of any of these.

As mentioned above, particularly preferred antioxidants are those that provide additional benefits to the skin such as ascorbyl palmitate. (See additional ingredients and methods in U.S. Pat. Nos. 4,775,530, 5,376,361, 5,409,693, 5,545,398, 5,574,063, 5,643,586, 5,709,868, 5,879,690, 5,965,618, 5,968,618, 6,051,244, 6,162,419, and 6,191,121 to Perricone).

Buffering agents may be desired. Preferably, the amount of buffering agent is one that results in compositions having a pH ranging from about 4.5 to about 8.5, more preferably from about 5.5 to about 8.5, most preferably from about 6.5 to about 8.0. Typical buffering agents are chemically and physically stable agents commonly found in cosmetics, and can include compounds that are also adjunct ingredients such as citric acid, malic acid, and glycolic acid buffers.

Some embodiments of this invention may contain at least one other adjunct ingredient in addition to glutathione. A preferable adjunct ingredient is lipoic acid, preferably alpha lipoic acid.

Alpha-lipoic acid (ALA) is expected to be a particularly effective adjunct ingredient. Oral administration of alpha-lipoic acid raises GSH levels in HIV patients, and is extremely safe and well tolerated. ALA is a broad-spectrum, fat- and water-phase antioxidant with potent electron-donating capacity, and has added biochemical versatility as a Krebs cycle cofactor and transition metal chelator. It is expected that the combination of glutathione and alpha lipoic acid will be particularly effective. A composition in accordance with this aspect of the invention might comprise 25% to 60% by weight glutathione and 0.5% to 5% by weight alpha lipoic acid.

As of the filing of this application, a preliminary program of clinical testing has been conducted. Patients exhibiting symptoms of psoriasis received a topical cream containing 45 mg/ml of reduced glutathione (GSH) in a phospolipid carrier. Patients instructed to apply the product twice daily to the affected areas. Patient lesions responded almost immediately to the treatment and were significantly improved, and in some cases had cleared, within 24 hours of the first application. In general, decreased inflammation, irritation, and erythema of the skin were observed. Elasticity and a supple feeling was returned to the skin.

The specific mechanisms for the beneficial effect of glutathione are not specifically understood at this time. However, I believe that glutathione reduces levels of inflammatory cytokines and transcription factors, as well as associated free radicals, and interrupts inflammatory cascade processes resulting in the regulation of the cell growth cycle. Accordingly, skin cells are produced in a normal manner instead of the accelerated and damaged state typical of psoriasis and other inflammatory skin conditions.

Insofar as has been determined based upon clinical studies to date, no adverse side effects are encountered.

Based on this data the preferred weight percentage specified herein have been projected as set forth above.

It is an advantage of the invention that compositions of the invention do not require a pharmaceutical prescription.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the claimed components and steps in any sequence which is effective to meet the objectives there intended, unless the context specifically indicates the contrary.

What is claimed is:

1. A composition for treatment of inflammatory skin conditions, comprising:
a carrier
reduced glutathione;
alpha lipoic acid.

2. A composition in accordance with claim 1, comprising:
25% to 60% by weight glutathione
0.5% to 5% by weight alpha lipoic acid.

3. A composition in accordance with claim 1, wherein said composition contains from 16% to 70% glutathione by weight.

4. A composition in accordance with claim 1, wherein said composition contains from 35% to 70% glutathione by weight.

5. A composition in accordance with claim 1, wherein said composition contains from 16% to 35% glutathione by weight.

6. A composition in accordance with claim 1, wherein said composition contains from 20% to 35% glutathione by weight.

7. A composition in accordance with claim 1, wherein said composition contains from 35% to 60% glutathione by weight.

8. A composition in accordance with claim 1, wherein said composition contains 45 milligrams of glutathione per milliliter of the carrier.

9. A composition in accordance with claim 1, wherein the carrier is a solution, dispersion, cream, lotion, gel or solid stick.
